(19) United States
(12) Patent Application Publication
(10) Pub. No.: US 2022/0301150 A1
(43) Pub. Date: Sep. 22, 2022

(54) Neutralization Estimating Apparatus and Neutralization Estimating Method

(71) Applicant: Nippon Telegraph and Telephone Corporation, Tokyo (JP)

(72) Inventors: Yosuke Takeuchi, Musashino-shi, Tokyo (JP); Ryuta Ishii, Musashino-shi, Tokyo (JP); Takuya Kamisho, Musashino-shi, Tokyo (JP); Masayuki Tsuda, Musashino-shi, Tokyo (JP)

(21) Appl. No.: 17/624,888

(22) PCT Filed: Jul. 11, 2019
(86) PCT No.: PCT/JP2019/027493

§ 371 Date: Jan. 5, 2022

(51) Int. Cl. G06T 7/00; G06T 7/11; G06T 7/90; G01N 21/88; G01N 33/38; G01N 21/27

(52) U.S. Cl. CPC G06T 7/0008; G06T 7/11; G06T 7/90; G01N 21/8851; G01N 33/383; G01N 21/27; G06T 2207/30132

(57) ABSTRACT

An estimation device has: determination unit for acquiring a characteristic color of reinforced concrete in a dry state from an initial image obtained by capturing an image of a concrete surface of the concrete, analyzing a first image of the concrete surface that is captured after spraying water onto the reinforced concrete, and determining a portion having a predetermined color difference from the characteristic color as a crack; and an estimation unit for impregnating the crack with water, analyzing a second image of the concrete surface that is captured after the concrete surface is dried, and estimating whether or not the inside of the crack is carbonated, using a color around the crack.

### TECHNICAL FIELD

The present invention relates to a carbonation estimation device and a carbonation estimation method for reinforced concrete.

### BACKGROUND ART

Reinforced concrete is a composite structure in which concrete protects reinforcing bars from corrosion. Since concrete is alkaline, reinforcing bars in sound reinforced concrete does not corrode. However, when a crack occurs in concrete, carbonation of the concrete proceeds from the crack portion, and corrosion of the reinforcing bars proceeds. For this reason, whether or not a crack has occurred in concrete and whether or not carbonation within the crack is proceeding is an important criterion for determining the soundness of reinforced concrete.

The presence of a crack is an inspection item that is mainly checked visually. In addition, a technology for determining a crack through image analysis has also been developed (see NPLs 1 and 2).

### CITATION LIST

### Non Patent Literature

[NPL 1] Kaneko, “Technology for Inspecting Crack in Concrete Structure Using Digital Camera Image”, NTT Technical Journal, (2011)
“Aim to Solve Social Issues by Making Full Use of AI: Challenges of Development Team of ‘THE JUDGE!’, Which Saves Labor in Inspection of Degree of Infrastructure Degradation”, [online], [Retrieved on Mar. 20, 2019], Internet <URL: https://blogos.com/article/356591/>

### SUMMARY OF THE INVENTION

### Technical Problem

Regarding corrosion of reinforcing bars, whether or not the inside of a crack in reinforced concrete is carbonated is an important criterion for determining the soundness of the reinforced concrete. However, to measure the carbonation state within a crack, execution of work accompanying destruction, such as breaking of a part of concrete, is required.

The present invention has been made in view of the foregoing problem, and an object of the present invention is to provide a technology for simply estimating carbonation of reinforced concrete without executing work to destroy the reinforced concrete.

### Means for Solving the Problem

To achieve the above object, a carbonation estimation device according to an aspect of the present invention includes: a determination unit for acquiring a characteristic color of reinforced concrete in a dry state from an initial image obtained by capturing an image of a concrete surface of the concrete, analyzing a first image of the concrete surface that is captured after spraying water onto the reinforced concrete, and determining a portion having a predetermined color difference from the characteristic color as a crack; and an estimation unit for impregnating the crack with water, analyzing a second image of the concrete surface that is captured after the concrete surface is dried, and estimating whether or not the inside of the crack is carbonated, using a color around the crack.

One aspect of the present invention is a carbonation estimation method performed by a carbonation estimation system, the method including: an acquisition step of acquiring a characteristic color of reinforced concrete in a dry state from an initial image obtained by capturing an image of a concrete surface of the concrete; a water spraying step of spraying water onto the reinforced concrete; a determination step of analyzing a first image of the concrete surface that is captured after spraying water onto the concrete surface, and determining a portion having a predetermined color difference from the characteristic color as a crack; an impregnation step of impregnating the crack with water; and an estimation step of analyzing a second image of the concrete surface that is captured after the water in the impregnation step has dried up, and estimating whether or not the inside of the crack is carbonated, using a color around the crack.

### Effects of the Invention

According to the present invention, it is possible to provide a technology for simply estimating carbonation of reinforced concrete without executing work to destroy the reinforced concrete.

### BRIEF DESCRIPTION OF DRAWINGS

FIG. 1 is a diagram showing an overall configuration of a carbonation estimation system.

FIG. 2 is a flowchart showing an operation of the carbonation estimation system.

FIG. 3 shows an example of a cross-section of a hollow concrete pole of reinforced concrete.

FIG. 4 shows examples of an image obtained when efflorescence has occurred and an image indicating an estimated carbonated region.

FIG. 5 is a diagram showing a configuration of a computer.

### DESCRIPTION OF EMBODIMENTS

Hereinafter, an embodiment of the present invention will be described with reference to the drawings. In the description of the drawings, same portions are assigned the same reference numerals, and a description thereof is omitted.

### (Overall Configuration of Carbonation Estimation System)

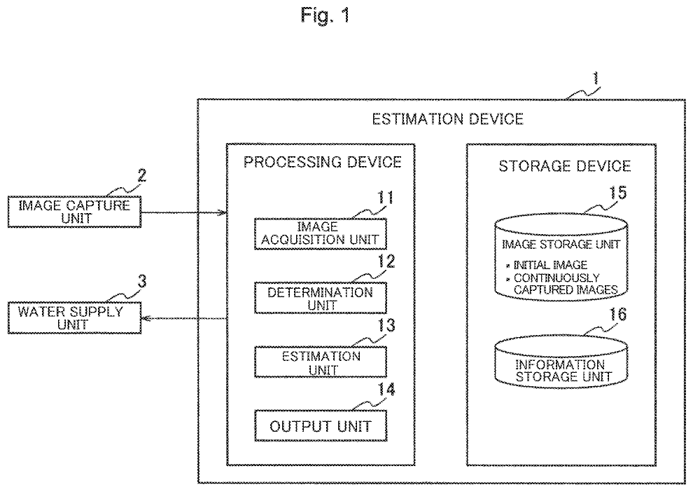

FIG. 1 is a diagram showing an overall configuration of a carbonation estimation system of the present embodiment. The carbonation estimation system estimates carbonation of reinforced concrete (reinforced concrete structure). In reinforced concrete, a plurality of reinforcing bars are arranged within concrete. The carbonation estimation system of the present embodiment includes an estimation device 1, an image capture unit 2, and a water supply unit 3.

The image capture unit 2 (image capture device) is a camera for capturing an image (still image or moving image) of a concrete surface of reinforced concrete. The image capture unit 2 is installed in a fixed manner at a position at which the image capture unit 2 can capture an image that enables a fine crack (e.g., with a width of about 0.1 mm) occurring on the concrete surface to be captured. The image capture unit 2 transmits the captured image to the estimation device 1.

The image capture unit 2 at least has a continuous image capture function, and may also have, for example, an interval image capture function, a function of taking out an image from a moving image at a predetermined timing, or the like. The image capture interval in interval image capture can be set to any period from one second to one hour.

In the present embodiment, the image capture unit 2 first transmits an initial image obtained by capturing an image of a concrete surface in a dry state to the estimation device 1. Thereafter, the image capture unit 2 transmits, to the estimation device 1, a plurality of still images obtained by performing interval image capture at predetermined intervals or a moving image as continuously captured images.

The water supply unit 3 (water supply device) supplies water to the concrete surface of reinforced concrete in accordance with a control performed by the estimation device 1. Note that, in the present embodiment, water is supplied by the estimation device 1 and the water supply unit 3, but rainfall may alternatively be utilized. Alternatively, a person may supply water to the crack.

The estimation device 1 analyzes the images of the concrete surface of reinforced concrete captured by the image capture unit 2, and estimates whether or not the inside of the crack is carbonated. The estimation device shown in the diagram has a processing device and a storage device. The processing device has an image acquisition unit 11, a determination unit 12, an estimation unit 13, and an output unit 14, and the storage device has an image storage unit 15 and an information storage unit 16.

The image acquisition unit 11 stores the images transmitted or distributed from the image capture unit 2 in the image storage unit 15. The image capture unit 2 transmits the initial image and the plurality of images (continuously captured images) that are captured using the continuous image capture function after capturing the initial image. The image acquisition unit 11 receives the initial image and the continuously captured images and stores the received images in the image storage unit 15.

The determination unit 12 acquires a characteristic color of the reinforced concrete in a dry state from the initial image of the concrete surface of the concrete, and stores the acquired characteristic color in the information storage unit 16. The determination unit 12 analyzes an image (first image) of the concrete surface that is captured during a drying process after water is sprayed onto the reinforced concrete, and determines a portion with a predetermined color difference from the characteristic color as a crack. Note that the determination unit 12 determines a crack using an image (first image) in which the proportion of a portion having the characteristic color exceeds a predetermined proportion, of the continuously captured images.

The estimation unit 13 controls the water supply unit 3 to cause the water supply unit 3 to impregnate the crack with water, analyzes an image (second image) of the concrete surface that is captured after the concrete surface is dried, and estimates whether or not the inside of the crack is carbonated, using the color around the crack. Specifically, to determine carbonation within the crack, the estimation unit 13 calculates the amount of water required to sufficiently impregnate the inside of the crack with water. The estimation unit 13 compares the color around the crack in the image (second image) obtained after the concrete surface is dried with the characteristic color, and estimates that the inside of the crack is not carbonated if the color around the crack has turned whiter than the characteristic color.

The estimation unit 13 analyzes a captured image (third image) of the concrete surface in an initial drying state after the impregnation, compares the color around the crack with the characteristic color, and estimates a region that has turned blacker than the characteristic color to be a carbonated region.

The output unit 14 outputs the determination result and the estimation result to an output device such as a display or a printer. The output unit 14 may also transmits the determination result and the estimation result to other devices via a network.

The image storage unit 15 stores the initial image and the plurality of images (continuously captured images) captured using the continuous image capture function. The information storage unit 16 stores various kinds of information such as the characteristic colors of concrete, a crack position, a crack width, the amount of water for the impregnation, the carbonation estimation result, and the carbonated region.

### (Operation of Carbonation Estimation System)

In the present embodiment, reinforced concrete is impregnated with neutral water with a pH of 7 to 8, whether or not the inside of a crack is carbonated is estimated by observing a color change during the drying process after the impregnation, and the carbonated region is estimated. The color of a concrete surface without a crack turns a slightly dark gray when wet, but water impregnation is less likely to proceed on such a concrete surface compared with a concrete surface in a portion where a crack has occurred (crack portion). For this reason, the concrete surface without a crack finishes being dried in a shorter time, and the color thereof returns to the original concrete color (gray). In contrast, a large amount of water remains on a concrete surface in the crack portion, and drying requires a longer time.

Therefore, in the drying process, there is the time in which water remains only on the concrete surface in the crack portion exists while most of the concrete surface is dry, and the crack portion is detected and carbonation is determined using this time. The color of the concrete surface without a crack in the dry state may be determined by acquiring, in advance, image information on the concrete before the water impregnation. The drying may be performed by leaving the concrete surface in the room temperature atmosphere, or the drying may be promoted by blowing warm wind thereto, for example.

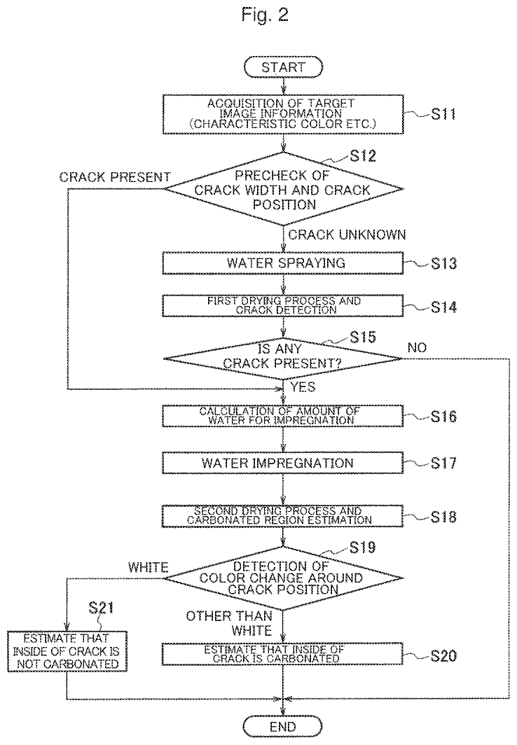

FIG. 2 is a flowchart showing an operation of the estimation system of the present embodiment.

In the present embodiment, the image capture unit 2 captures an image of a target concrete surface of reinforced concrete using the continuous image capture function, and transmits the captured image to the estimation device 1. The image capture unit 2 of the present embodiment transmits a plurality of images captures through interval image capture at predetermined image capture intervals (e.g., of one second to one hour) to the estimation device 1. Note that the image capture unit 2 may a web camera or the like, capture a video (moving image) of the concrete surface, and distribute the captured video to the estimation device 1. In this case, the estimation device 1 may take out still images from the distributed video at predetermined image capture intervals, store the still images in the image storage unit 15, and analyze these images.

First, the estimation device 1 receives an initial image of an initial (first) state transmitted from the image capture unit 2, analyzes the initial image, and extracts a characteristic color of the concrete in the dry state (S11). Note that the estimation device 1 makes the characteristic color into digital information indicating RGB or CMYK, and stores the digital information in the information storage unit 16. Note that the estimation device 1 stores the image transmitted from the image capture unit 2 in the image storage unit 15.

If a crack (crack position, crack width) on the concrete surface can be visually recognized in advance (S12: crack present), the estimation device 1 proceeds to S16 and transitions to carbonation estimation by means of water impregnation. In this case, an operator inputs the checked crack position and crack width to the estimation device 1, and stores the checked crack position and crack width in the information storage unit 16.

If a crack on the concrete surface cannot be recognized in advance (S12: crack unknown), the estimation device 1 proceeds to S13 and visualizes a crack by spraying water. This is because, if a minute crack (with a width of 0.1 mm or less) is present, such a crack is difficult to visually recognize.

When a crack is visualized, the estimation device 1 transmits a control signal to the water supply unit 3. The water supply unit 3 sprays water onto the target concrete surface in accordance with the control of the estimation device 1 (S13). Water captured at an opening portion of a crack enhances absorption of light at the position at which the water is captured. Thus, by analyzing the captured images of the concrete surface, the estimation device 1 can detect a portion having a darker color than the characteristic color of dry concrete as a crack portion, and estimate the width of this darker portion to be the crack width.

The water supply unit 3 need only spray water in the manner that an atomizer sprays water, and need only spray water to the extent that the target concrete surface is sufficiently wet, that is, such that a water film with a thickness of about 0.1 mm is formed on the surface area of a target area.

Thereafter, the operation transitions to a first drying process (S14). The drying may be performed by leaving the concrete surface in the room temperature atmosphere, or the drying may be promoted by blowing warm air wind using a drying device (not shown).

In the first drying process, the estimation device 1 analyzes the images transmitted from the image capture unit 2 at the predetermined image capture intervals, and determines whether or not a crack is present using an image (first image) that has become close to the initial image when the concrete surface dries (S14). The image that has become close to the initial image is an image in which the characteristic color of the initial image exceeds a predetermined proportion (e.g., 90%).

If a portion in which a predetermined color difference from the characteristic color occurs is present in the image that has become close to the initial image, the estimation device 1 determines that a crack is present. If a portion in which the predetermined color difference from the characteristic color is not present, the estimation device 1 determines that a crack is not present. That is to say, the estimation device 1 detects, as a crack portion, a portion having a darker color than the characteristic color of the dry concrete that is acquired in S11, and estimates the width of this darker portion (thickness of a line in the darker portion) to be the crack width. If it is determined that a crack is present, the estimation device 1 acquires the position of the detected crack and the width of the crack by analyzing the image, and stores the position and the width of the crack in the information storage unit 16.

If a crack is not present (S15: no), the estimation device 1 ends the processing. If a crack is present (S15: yes), the estimation device 1 calculates the amount of water required to sufficiently impregnate the inside of the crack with water, in order to determine carbonation within the crack (S16). The estimation device 1 calculates the minimum required amount of water using the crack width acquired in S14 and a crack depth estimated from the structure of reinforced concrete, and sets the calculated minimum required amount of water or more as the amount of water for the impregnation. Since the minimum required amount of water or more may be set as the amount of water for the impregnation, the crack depth may be the largest estimated depth of the crack, e.g., the entire length of the concrete thickness of the structure.

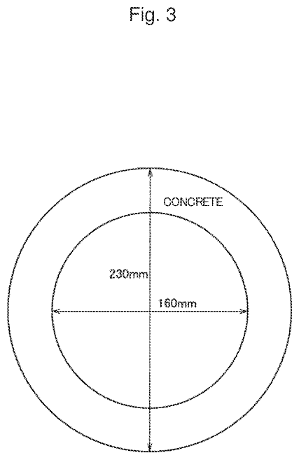

For example, if a crack with a width of 0.1 mm has occurred in a half circumference of hollow concrete shown in FIG. 3, the estimation device 1 calculates the area (107 cm²) of this crack region using the following equation.

{(23/2)²×π−(16/2)²×π}/2=107 cm²

The estimation device 1 then multiplies the area (107 cm²) of the crack region by the crack width (0.01 cm) and calculates the crack space (107 cm²×0.01 cm=1.07 cm³). Since a space of about 1.07 cm³ has occurred, the estimation device 1 figures out 1.07 g as the sufficient amount of water to fill this space.

In the present embodiment, the entire target concrete surface is impregnated with water, similarly to the water spraying in S13. Therefore, the estimation device 1 calculates the total amount of water for the impregnation by multiplying a value per unit area (cm²) (here, about 3 g/cm²) by the area of the concrete surface to be measured. The value per unit area (about 3 g/cm²) is a value obtained by multiplying the length (23×π/2=36.13 cm) of the half circumference of the concrete by the crack with a width of 0.1 mm (36.13×0.01=0.36 cm²), and dividing the amount of water for filling the crack space by the above-multiplied value (1.07 g/0.36 cm²=2.96 g/cm²).

Note that if the water supply unit 3 has a mechanism for impregnating only the crack region with water, the aforementioned 1 g may be calculated as the amount of water.

The estimation device 1 transmits a control signal including the calculated amount of water for the impregnation to the water supply unit 3. The water supply unit 3 impregnates the target concrete surface with the amount of water for the impregnation in accordance with the control performed by the estimation device 1 (S17). Note that, as for reinforced concrete installed outdoors, water impregnation using rainfall or the like may also be utilized. If only the crack region is impregnated with water, the estimation device 1 may include the crack position in the control signal.

After the entire concrete surface including the crack region are sufficiently impregnated with water, the operation proceeds to a second drying process (S18). The drying may be performed by leaving the concrete surface in the room temperature atmosphere, or drying may be promoted by blowing warm air wind using a drying device (not shown).

Concrete components dissolve in the water within the crack from the inside of the crack. At this time, if the inside of the crack is in a non-carbonated state, calcium hydroxide dissolves in the water. The water in which calcium hydroxide dissolves leaks to the concrete surface outside the crack, and thereafter the water evaporates. As a result, the concrete surface around the crack portion enters a state where a white deposit called efflorescence occurs.

On the other hand, if the inside of the crack is carbonated, calcium hydroxide changes to calcium carbonate within the crack, and the solubility thereof in water decreases. For this reason, the efflorescence components do not dissolve in the water, and neutral water leaks to the concrete surface outside the crack and evaporates. In a region in a state where neutral water is retained in the crack portion, the amount of light reflected decreases, and thus, this region appears blacker than the other region.

If the crack portion is carbonated, the crack portion is in a state after alkaline components contained in the concrete have leaked out, and therefore the crack portion is in a rougher state than concrete in a non-carbonated region and firmly retains water. For this reason, the concrete surface also firmly retains water in the carbonated region around the crack, and discoloration to black is observed. Therefore, the portion that has been discolored black can be estimated to be a carbonated region.

Thus, in the second drying process, it is possible to determine, based on a white deposit, whether or not alkaline components are contained when water exudes from the crack portion to the concrete surface and evaporates. Accordingly, the presence of a white deposit enables determination of carbonation within the crack.

In the second drying process after the impregnation, the estimation device 1 analyzes a captured image (third image) of the concrete surface, compares the color around the crack with the characteristic color of the concrete, and estimates a region that has turned blacker than the characteristic color to be a carbonated region (S18).

Specifically, similarly to the first drying process in S14, the estimation device 1 analyzes the images (continuously captured images) transmitted from the image capture unit 2 at the predetermined image capture intervals, and estimates a carbonated region using an image (third image) that has become close to the initial image when the concrete surface is dried. The image that has become close to the initial image is an image in which the characteristic color of the initial image exceeds a predetermined proportion (e.g., 90%).

On the concrete surface that has been impregnated with water and then begun to be dried, water evaporation proceeds, and a state is entered where the water captured in the crack is left as in S14. Since efflorescence occurs at a stage when the evaporation of water within the crack has proceeded, the crack is detected as a black portion in the image (third image) that has become close to the initial image obtained in the second drying process. Since the concrete surface has been impregnated with the sufficient amount of water in S17, the estimation device 1 analyzes this image, temporarily estimates a portion with a predetermined color difference from the characteristic color (i.e., a portion blacker than the characteristic color) to be a carbonated region, and stores the estimation result in the information storage unit 16 (S18).

Thereafter, when the drying has sufficiently proceeded, efflorescence can be determined. At this time, the estimation device 1 analyzes an image (second image) of the concrete surface captured after the impregnated water has dried up, and estimates whether or not the inside of the crack is carbonated, using the color around the crack. That is to say, the estimation device 1 estimates carbonation within the crack using an image in a state where the impregnated water has also evaporated within the crack and the inside of the crack is dry. The image (second image) after the drying is an image in which the color around the crack is the same as the characteristic color of the concrete surface, or white, of the plurality of continuously captured images.

Image analysis using color is described in NPL 1, for example. In the present embodiment, the estimation device 1 compares the color around the crack in the image (second image) with the characteristic color of the concrete surface (S19).

For example, if a color is expressed by a value of 0 to 255 in a grayscale display with a dark background, the estimation device 1 determines a color as white if the value of the color is greater than the characteristic color of the concrete by 50 or more, and determines a color as black if the value of the color is smaller than the characteristic color of the concrete by 30 or more.

If the around the crack has turned whiter than the characteristic color (S19: white), the estimation device estimates that the inside of the crack is not carbonated (S21). In this case, the estimation device 1 discards the carbonated region estimated in S18. On the other hand, if the color around the crack is other than white (S19: other than white), the estimation device estimates that the inside of the crack is carbonated, and regards the region estimated in S18 as a carbonated region (S20). The estimation device 1 stores the estimation result in the information storage unit 16.

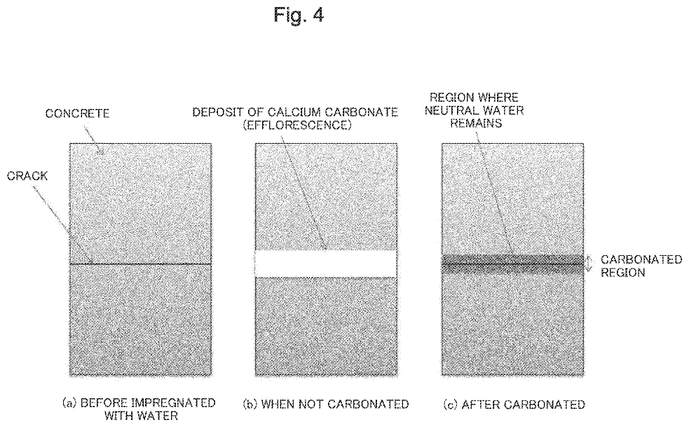

FIG. 4 is a schematic diagram of an image in which efflorescence, which indicate a non-carbonated state, has occurred in the crack on the concrete surface, and an image indicating an estimated carbonated region. FIG. 4(a) indicates the position of a crack on the concrete surface. FIG. 4(b) shows an example image in the case where efflorescence, which indicates a non-carbonated state and is a deposit of calcium carbonate, has occurred around the position of the crack. FIG. 4(c) shows an example image in the case where neutral water remains around the position of the crack, which has blackened. The estimation device 1 estimates this blackened portion to be a carbonated region.

Note that if past efflorescence already appears on the concrete surface, it is difficult to distinguish the past efflorescence from efflorescence detected according to the present embodiment, and it is therefore desirable that the past efflorescence is removed in advance.

The estimation device 1 of the above-described present embodiment has the determination unit 12 and the estimation device 13. The determination unit 12 acquires a characteristic color of reinforced concrete in a dry state from an initial image obtained by capturing an image of a concrete surface of the concrete, analyzes a first image of the concrete surface that is captured after spraying water onto the reinforced concrete, and determines a portion with a predetermined color difference from the characteristic color as a crack. The estimation unit 13 analyzes a second image of the concrete surface that is captured after impregnating the crack with water and drying the concrete surface, and estimates whether the inside of the crack is carbonated, using the color around the crack.

That is to say, a crack portion in reinforced concrete firmly captures water, and a significant color change occurs compared with a portion without a crack. The water captured by the crack evaporates on the surface of the concrete, and an internal carbonated state can be estimated based on the presence of a white deposit. For this reason, in the present embodiment, non-destructive and simple carbonation determination can be realized by determining the color of a crack portion during a drying process after water impregnation.

In the present embodiment, whether a crack is carbonated or not carbonated can be determined, and it is therefore possible to determine the soundness of a reinforced concrete structure after a crack has occurred, and to realize appropriate facility maintenance.

In the present embodiment, the estimation device 1 analyzes a third image obtained by capturing an image of the concrete surface in an initial drying state after the impregnation, compares the color around the crack with the characteristic color, and estimates a region that has turned blacker than the characteristic color to be a carbonated region.

The size of the carbonated region on the concrete surface indicates the largest carbonated region in the depth direction of the crack, and can therefore be regarded as the largest corrosion area of metal buried in the concrete. When metal corrosion in concrete is electrochemically investigated, it is necessary to clarify the corrosion area, especially for evaluation of the corrosion rate. In the present embodiment, information that can be used to estimate the corrosion rate can be obtained.

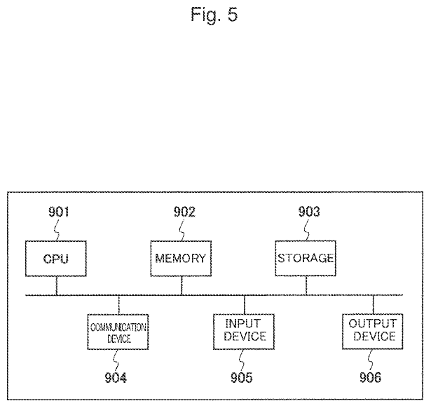

Note that, for example, a general-purpose computer system such as that shown in FIG. 5 can be used as the above-described estimation device 1. The shown computer system includes a CPU (Central Processing Unit, processor) 901, a memory 902, a storage 903 (HDD: Hard Disk Drive, SSD: Solid State Drive), a communication device 904, an input device 905, and an output device 906. The memory 902 and the storage 903 are storage devices. In this computer system, the functions of the estimation device 1 are realized by the CPU 901 executing a predetermined program loaded to the memory 902. The estimation device 1 may be realized by one computer, or may be realized by a plurality of computers. Further, the estimation device 1 may be a virtual machine implemented on a computer.

The program of the estimation device 1 can be stored in a computer-readable recording medium such as an HDD, an SSD, a USB (Universal Serial Bus) memory, a CD (Compact Disc), or a DVD (Digital Versatile Disc), and can also be distributed via a network.

The present invention is not limited to the above-described embodiment, and can be modified in various manners within the scope of the gist of the invention.

### REFERENCE SIGNS LIST

1: Estimation device
11: Image acquisition unit
12: Determination unit
13: Estimation unit
14: Output unit
15: Image storage unit
16: Information storage unit
2: Image capture unit
3: Water supply unit

1. A carbonation estimation device comprising:
a determination unit for acquiring a characteristic color of reinforced concrete in a dry state from an initial image obtained by capturing an image of a concrete surface of the concrete, analyzing a first image of the concrete surface that is captured after spraying water onto the reinforced concrete, and determining a portion having a predetermined color difference from the characteristic color as a crack; and
an estimation unit for impregnating the crack with water, analyzing a second image of the concrete surface that is captured after the concrete surface is dried, and estimating whether or not the inside of the crack is carbonated, using a color around the crack.

2. The carbonation estimation device according to claim 1, wherein the estimation unit compares the color around the crack in the second image with the characteristic color, and estimates that the inside of the crack is not carbonated if the color around the crack has turned whiter than the characteristic color.

3. The carbonation estimation device according to claim 1, wherein the first image is an image in which the proportion of a portion having the characteristic color exceeds a predetermined proportion.

4. The carbonation estimation device according to claim 1, wherein the estimation unit analyzes a third image of the concrete surface that is captured during a drying process after the impregnation, compares the color around the crack with the characteristic color, and estimates a region that has turned blacker than the characteristic color to be a carbonated region.

5. A carbonation estimation method performed by a carbonation estimation system, the method comprising:
an acquisition step of acquiring a characteristic color of reinforced concrete in a dry state from an initial image obtained by capturing an image of a concrete surface of the concrete;
a water spraying step of spraying water onto the reinforced concrete;
a determination step of analyzing a first image of the concrete surface that is captured after spraying water onto the concrete surface, and determining a portion having a predetermined color difference from the characteristic color as a crack;
an impregnation step of impregnating the crack with water; and
an estimation step of analyzing a second image of the concrete surface that is captured after the water in the impregnation step has dried up, and estimating whether or not the inside of the crack is carbonated, using a color around the crack.

6. The carbonation estimation method according to claim 5, wherein the estimation unit compares the color around the crack in the second image with the characteristic color, and estimates that the inside of the crack is not carbonated if the color around the crack has turned whiter than the characteristic color.

7. The carbonation estimation device according to claim 2, wherein the first image is an image in which the proportion of a portion having the characteristic color exceeds a predetermined proportion.

8. The carbonation estimation device according to claim 2, wherein the estimation unit analyzes a third image of the concrete surface that is captured during a drying process after the impregnation, compares the color around the crack with the characteristic color, and estimates a region that has turned blacker than the characteristic color to be a carbonated region.

9. The carbonation estimation device according to claim 3, wherein the estimation unit analyzes a third image of the concrete surface that is captured during a drying process after the impregnation, compares the color around the crack with the characteristic color, and estimates a region that has turned blacker than the characteristic color to be a carbonated region.
